(19) United States
(12) Patent Application Publication
(10) Pub. No.: US 2023/0034882 A1
(43) Pub. Date: Feb. 2, 2023

(54) LASER DEVICE FOR SKIN TREATMENTS AND METHOD

(71) Applicant: EL.EN. S.P.A., Calenzano, FI (IT)

(72) Inventors: Leonardo MASOTTI, Sesto Fiorentino (IT); Riccardo STOCCHI, Seano (IT); Marco TAGLIAFERRI, Calenzano (IT); Tiziano ZINGONI, Calenzano (IT)

(21) Appl. No.: 17/775,816

(22) PCT Filed: Nov. 11, 2020
(86) PCT No.: PCT/IB2020/060604

§ 371 Date: May 10, 2022

(30) Foreign Application Priority Data
Nov. 11, 2019 (IT) 102019000020703
Jul. 15, 2020 (IT) 102020000017173

(51) Int. Cl. A61N 5/067; A61N 5/06

(52) U.S. Cl. CPC A61N 5/067; A61N 5/0616; A61N 2005/0644; A61N 2005/0631; A61N 2005/005

(57) ABSTRACT

The device includes a laser source (7) adapted to emit a laser radiation at a wavelength between around 620 nm and around 750 nm, and a handpiece (5). The handpiece in turn includes an applicator (11) with a contact surface with the epidermis defining a window (11.1) for the passage of a laser beam (F) toward the epidermis (E) of a subject to be treated. A waveguide (10) conveys the laser radiation from the laser source (7) to a scanning system (17) of the handpiece.

### TECHNICAL FIELD

Disclosed herein are devices and methods for laser skin treatment. In particular, devices and methods disclosed herein are used to treat imperfections of the skin, such as wrinkles, benign pigmented marks and scars, for specifically cosmetic purposes.

### BACKGROUND ART

In the cosmetic treatment sector, the use of laser sources to perform various types of operations on live tissues is known. Some of these operations concern skin treatment, in order to remove imperfections or alterations, in particular caused by age.

The use of non-ablative lasers, in particular in the near infrared (NIR) range with wavelengths between 1320 and 1540 nm, is known for rejuvenation treatments or other skin treatments up to the dermal level. However, these lasers cause damage, albeit minimum, to the epidermal layer, requiring relatively long post-op recovery times, with discomfort for the patient.

WO00/53261 discloses a device for the treatment of live tissue and in particular for the treatment of the epidermis. In some embodiments use of a laser source is suggested. These include Nd:YAG laser, used with a dual frequency output, at a wavelength between 520 nm and 680 nm. This source at these wavelengths is recommended for treating hypervascular lesions. In this application, the laser is used to cause blood photocoagulation, without interfering with the surrounding tissue. Therefore, the wavelengths selected are aimed at reaching a high affinity with (high absorption by) hemoglobin. The power, energy, energy density and fluence ranges are aimed at obtaining photocoagulation. The same publication also mentions the use of laser for tissue ablation. In this case, the wavelengths selected such as to be absorbed by water present in the cells. Wavelengths between 700 and 900 nm, and in particular about 810 nm, with appropriate powers and energy densities to cause ablation, are suggested.

US2019/0201705 discloses a cosmetic treatment system of biological structures using magnetic fields. In this context, combining a treatment with variable magnetic fields with light energy delivery to obtain a generic effect on the tissues treated is generically suggested. The publication mentions a plurality of light sources, both of coherent and non-coherent light and indicates a wavelength range variable from 190 to 13000 nm, without providing any specific correlation between wavelength and interaction with specific tissues. In general, wavelengths from 915 nm upward are preferred. Other emission parameters remain generic and not aimed at achieving specific interactions with given components of the tissues under treatment.

EP3246069 discloses a medical device for laser therapy. The device contains a combination of laser sources that emit at different wavelengths in a range between 550 nm and 1075 nm, in power ranges indicated in a very variable manner from 100 mW to 15 W. No specific teachings are provided on the energy delivery methods and on the fluences used, or on the methods of spatially controlling the laser spot of the single laser beams.

US2013/0041309 discloses an apparatus and a method for photo-stimulation, for photo-dynamic therapy and for ablative laser treatments of biological tissue. The teaching provided by this document is very generic. The sources used are indicated as having powers in a range from 0.1 mW to 25,000 mW and with wavelengths from 400 nm to 3000 nm.

Wrinkles are one of the main clinical alterations of skin associated with skin aging. The dermal layer of the skin contains oriented and organized collagen fibers, which contribute to the tone and elasticity of the dermis. Skin aging is a multi-factorial process depending both on intrinsic factors (of genetic, hormonal and metabolic nature), and on extrinsic factors (long term exposure to UV radiation, air pollution, smoking, poor diet, contact with chemical products).

Intrinsic skin aging is genetically determined and involves various genes. A genetic defect can cause telomerase insufficiency and condsequent arrest of the cells in replication of the stratum basale, causing thinning of the epidermis. Intrinsic skin aging also comprises an alteration of elastin, fibrillin and oligosaccharides. Estrogen deficiency, typical in menopause, leads to a rapid increase in skin aging. Hypoestrogenism causes a decrease in the production of hyaluronic acid and consequent reduction in skin viscosity. The skin, above all of the face, becomes thin, dry and wrinkled.

Exposure to UV radiation is the primary factor of extrinsic skin aging. Exposure to the sun mainly affects the stratum corneum, causing thickening of the skin due to a reduction of the expression of type I collagen and of type VII collagen in the keratinocytes. Type VII collagen forms the anchoring fibrils to the dermal-epidermal interface. This reduction contributes to wrinkles formation due to the weakened connection between dennis and epidermis and abnormal accumulation of elastic tissue in the dermis.

Degradation of collagen and abnormal accumulation of elastin in the dermis can lead to a loss of thickness and elasticity with the formation of wrinkles.

The treatment of facial wrinkles has become one of the main topics of cosmetic dermatology.

The development of non-surgical procedures, such as the use of toxins, fillers and chemical peeling, has reduced surgical procedures and leads patients to give preference to these simpler treatments.

The treatment of facial wrinkles requires the use of different types of ablative and non-ablative lasers. Ablative lasers are absorbed by water in the dermis causing immediate vaporization thereof, while non-ablative lasers cause it to heat, thus coagulating and heating the dermis and consequently stimulating fibroblast activity in order to produce new collagen. Non-ablative treatments have fewer side effects and reduce healing times. However, they can require several treatment sessions to achieve the desired result, in particular in the treatment of deep wrinkles.

In the field of ablative lasers, both CO₂ lasers and Er:YAG micro-ablative lasers, use water as chromophore for the transfer of energy to the collagen fibers, as do non-fractional and non-ablative systems in the near infrared range. This phase, although effective, requires a more complex post-op management of the dermis and of the epidermis.

It would therefore be beneficial to have a device and a method that allow less invasive dermal treatments, in particular for cosmetic purposes, without epidermal damage and with easier and shorter post-treatment recovery times, so as to result in less discomfort for the patient.

### SUMMARY

It has been discovered, and is relevant for the purposes described herein, that with a skin treatment using laser radiation in the red range it is possible to overcome or greatly reduce the problems of prior art methods. Studies conducted by the present inventors have shown that with radiation in the red range, in particular combined with a scanning system for the treatment of micro-areas through stimulation by means of a sub-ablative and selective heat treatment of the skin, it is possible to obtain important results in terms of reduction of many skin imperfections, in particular reduction of wrinkles, reducing or eliminating the unpleasant side effects of known treatments. Devices and methods that associate laser treatment with cooling of the area of epidermis under treatment are particularly effective.

Specifically, the range of wavelengths that can be used is preferably selected so as to have high affinities with the collagen fibers and a minimum of interaction with the vascular components of the volume of tissues under treatment. In this way, the laser radiation acts directly on the collagen component of the tissues avoiding absorption by the water and by other chromophores present in the tissues, thus transferring thermal energy selectively to the collagen fibers and reducing the involvement of other chromophores.

A device for skin treatment, has been provided, comprising: a laser source adapted to emit a laser radiation at a wavelength between about 620 nm and about 750 nm; a handpiece, comprising: an applicator with an epidermis contacting surface defining a window for the passage of a laser beam toward the epidermis; a waveguide adapted to convey laser radiation from the laser source to the handpiece. In some embodiments, the handpiece comprises a laser beam scanning system, for application of the laser beam in areas of the epidermis arranged according to a pattern that can be set by the operator or selected, for example, from patterns previously stored in a library.

More specifically, the laser source used has a wavelength preferably between about 635 nm and about 715 nm, more preferably between about 635 and about 700 nm, even more preferably between about 650 and about 700 nm. In some embodiments, a laser source with a wavelength about 675 nm, or about 694 nm, is used.

The device can have a scanning system with a scanning area with sizes between about 10 and about 25 mm, for instance. For example, the scanning area can be a square area with a side between 10×10 mm and 25×25 mm, for example typically 15×15 mm. It would also be possible to use elliptical or circular scanning areas, with major axis or a diameter comprised, for example, between about 10 mm and about 25 mm, typically about 15 mm.

The device is adapted to generate micro-areas of heating and consequent thermal, sub-ablative and selective stimulation on the skin, preserving the epidermal layer due to a cooling system integrated in the handpiece, adapted to maintain a temperature of the cooling means about 5° C., for instance.

The selected wavelength is particularly advantageous due to the absence of, or limited interaction with, hemoglobin (low hemoglobin absorption). This, advantageously in combination with adequate cooling of the epidermis under treatment, can greatly reduce inflammatory reaction and can also substantially reduce the risk of post-treatment pigmentations.

In some embodiments, the handpiece further comprises a video camera for taking images of the treated area. Images can be taken through the treatment window, so that the operator can carefully observe the area in which the handpiece is applied, even if this area is covered by the handpiece. A protective system, for example an optical or mechanical shutter, can protect the sensor of the video camera from the laser beam. For this purpose, the shutter can be synchronized with the laser emission. If the laser is a pulsed laser, images are taken between one pulse and the next, while during emission the sensor of the video camera is shielded, to avoid damage thereto caused, for example, by laser radiation backscattered from the epidermis, or from an element of the handpiece.

The handpiece can be equipped with a suitable local cooling system of the skin treated. Examples of cooling systems are described below.

The laser source can be a solid-state source. In some embodiments, the laser source comprises a laser diode or a plurality of laser diodes.

In embodiments described herein, in particular, the device comprises a laser source with a wavelength between about 635 or about 715, preferably about 675 nm or about 694 nm, with a power between about 0.5 W and 20 W, preferably between about 0.5 W and about 10 W, and a scanning system and a lens that apply on the surface to be treated spots with sizes that, with a dwell time of each spot between about 0.1 seconds and 2 seconds, preferably between 0.1 seconds and 0.5 seconds, irradiate an amount of energy between about 0.1 Joules and about 10 Joules, preferably between about 0.25 Joules and about 5 Joules, with a fluence between about 51 J/cm² and about 2550 J/cm², preferably between about 100 J/cm² and about 1100 J/cm².

Also suggested herein is a method for cosmetic treatment for removing or reducing skin imperfections of a subject, comprising the step of irradiating a portion of epidermis of the subject being treated with a laser beam at a wavelength between about 620 nm and about 750 nm, preferably between about 635 nm and about 715 nm, preferably between about 635 and about 700 nm, preferably between about 650 and about 700 nm, and more preferably about 675 nm, or about 694 nm.

Further advantageous features and embodiments of the device and of the method are described and defined in the appended claims, which form an integral part of the present description.

In the description and in the appended claims the term “about” is meant as an approximation of +/−15%, preferably +/−10%, more preferably +/−2%, even more preferably +/−1%, or +/−0.5% of the value indicated. Typically, for example in the case of diode laser sources, the term “about” referred to a specific wavelength value is meant in the sense that the value indicated is inclusive of dispersion of the emission characteristics typical of these types of laser sources.

### BRIEF DESCRIPTION OF THE DRAWINGS

The invention will be better understood by perusal of the description and the accompanying drawings, which illustrate a non-limiting exemplary embodiment of the invention. More in particular, in the drawings:

FIG. 1 shows a diagram of the device;

FIG. 2 shows a schematic section of a skin portion;

FIG. 3 shows a diagram of a treatment pattern;

FIGS. 4 to 15 show microphotographs of biopsies illustrating the results obtained with a device and method disclosed herein; and

FIG. 16 shows a diagram of the absorption coefficient as a function of the wavelength of different components contained in the epidermis.

### DETAILED DESCRIPTION

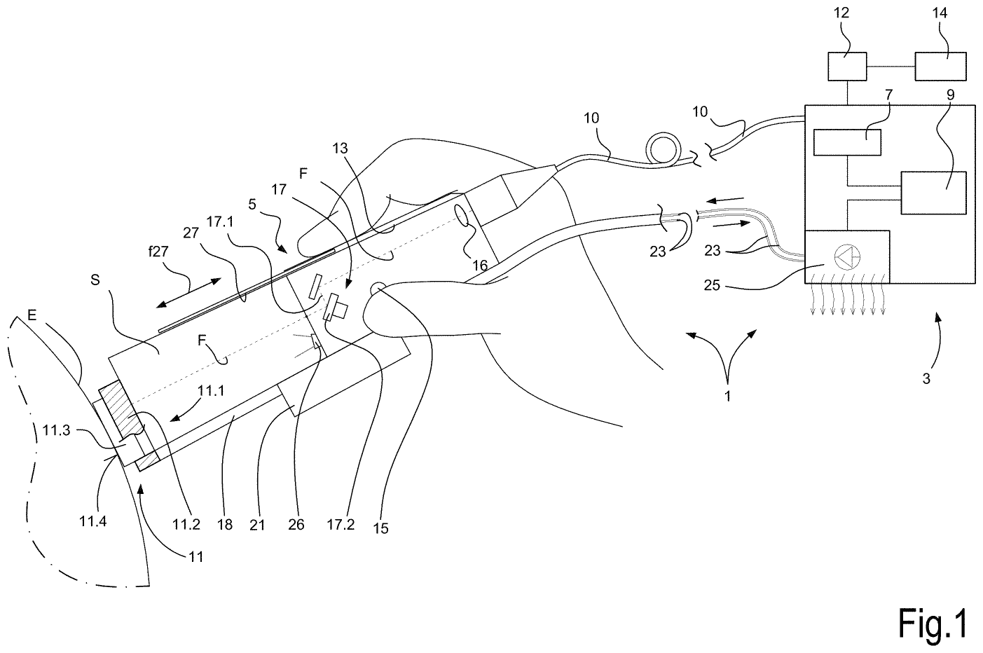

FIG. 1 schematically represents a device 1 for skin treatment using laser in the red range. The device 1 comprises an apparatus 3 and a handpiece 5. In the diagram of FIG. 1, the handpiece 5 is represented greatly enlarged with respect to the apparatus 3 for reasons of clarity of representation. The two parts of the drawing, relating to the handpiece and to the apparatus, are therefore in different scales.

The apparatus 3 can house a laser source 7, for example a laser diode or a plurality of combined laser diodes. Characteristically, the laser source 7 emits at a wavelength in the visible range. Specifically, the laser source 7 emits in the red range, with a wavelength between about 620 nm and about 750 nm. In currently preferred embodiments, the emission wavelength of the laser source 7 is between about 635 nm and about 715 nanometers, and in an embodiment can be about 675 nm or about 694 nm.

The reference number 9 generically indicates a central control unit connected to the laser source 7 to control the emission parameters of the laser source 7. The central control unit 9 can be interfaced with the handpiece 5, in particular to receive commands or signals therefrom.

The apparatus 3 is connected to the handpiece 5 through a waveguide, for example an optical fiber 10, which conveys the laser radiation generated by the laser source 7 toward the handpiece 5.

The handpiece 5 can comprise a contact structure for contacting a patient, i.e., an applicator 11 configured to be brought into contact with the epidermis of the patient being treated. The applicator 11 can be connected with a portion of a handle 13 of the handpiece 5. The handle 13, which can be shaped ergonomically for ease of use, can contain one or more buttons or other control members, one of which is generically indicated with 15. The control members can be interfaced with the central control unit 9 through any suitable communication channel, for example through wiring, not shown.

The handpiece 5 can be supplied with power through a cable, or can be powered by a battery, optionally rechargeable.

The handpiece 5 can comprise a scanning system for moving a laser beam generated by the laser source 7 according to a suitable pattern under the control of the central control unit 9. The diagram of FIG. 1 represents a scanning system 17 that can have a mirror 17.1 positioned in front of a collimator or focusing lens, schematically indicated with 16, positioned at the outlet of the optical fiber 10. The mirror 17.1 can be stationary with respect to the handpiece 5 and can divert the laser beam F delivered from the optical fiber 10 toward a moving mirror 17.2. This latter can be controlled by an actuator 17.3 to move the laser beam F according to a previously selected pattern. The pattern can be selected, for example, from a series of possible patterns stored in the central control unit 9, or can be loaded into the central control unit 9 before starting a laser session. In other embodiments, the operator can set some characterizing parameters of the pattern and the central control unit 9 can be programmed to calculate the pattern based on the parameters entered by the operator. To facilitate these and other operations, the apparatus 3 can be provided with a monitor 12 and with a human-machine interface (HMI) schematically shown at 14, which can in turn comprise several devices, such as a mouse, a keyboard, a touchscreen, or the like.

The laser beam F output from the optical fiber 10 can act on the epidermis E of the patient passing through the applicator 11. This latter can comprise a window 11.1 made of a material transparent to the wavelength of the laser radiation coming from the laser source 7. In some cases, the device can comprise a video camera and/or a thermal camera to allow viewing of the area being treated and/or to detect the temperature of the skin in the area being treated, during application of the laser radiation. In this case, as the video camera or thermal camera is positioned behind the window 11.1, advantageously this should be transparent to the radiation used for the viewing system.

In particular, with a laser source 7 that emits in the red range, the material of the window 11.1 can be a sapphire block 11.3. The sapphire block 11.3 can, for example, have a cylindrical shape and can be inserted in a frame 11.2. Reference number 11.4 indicates the outer surface of the sapphire block 11.3, which forms the contacting surface for contacting the epidermis E of the patient to be treated. If the sapphire block 11.3 is cylindrical in shape, the frame 11.2 can be annular in shape.

In other embodiments, in particular in order to use a video camera and/or a thermal camera, the window can be free, i.e., open and without a closing material, so as not to obstruct viewing in the infrared and/or in the visible range through the thermal camera and/or the video camera. The window can in this case, for example, be surrounded by a frame, which defines an epidermis contacting surface of the handpiece.

As will be clarified below, the laser treatment of the skin of the patient can benefit from cooling performed before and/or during treatment. In principle, cooling can be performed extemporaneously through cooling means separate from the treatment device 1, for example by directing a jet of cold air or other cold gases onto the portion of skin to be treated. However, this mode of operation is not advantageous and is somewhat impractical.

In some embodiments, the applicator 11 can be associated with a cooling system, or a cooling system can be integrated in the applicator 11 and/or in the handpiece 5.

An air cooling system can be provided, for instance, which guarantees a repeatable and effective cooling process. Uniform air cooling of the whole of the area to be treated can be achieved by means of meticulous design of the nozzle that models the air jet and by ensuring that the cooling parameters (temperature and flow) are maintained constant by a suitable measurement and feedback system.

Moreover, the use of the air cooling system has the great advantage of facilitating the use of a thermal camera that measures the skin temperature in real time, both in the cooling process (pre-treatment), and during laser emission, in this way making it possible to monitor the maximum local temperature of each spot or dot treated in order to guarantee treatment safety.

With real time temperature control, it is possible to work on each spot with a sequence of pulses, the number whereof can be determined automatically for each spot in relation to reaching a target temperature value associated with safety and efficacy of the treatment.

Typically, when an air cooling system is used, the handpiece will have an epidermis contacting surface defined, for example, by a frame that delimits an open window, i.e., without mechanical closing members, which can instead be used typically in the case in which cooling takes place by means of a liquid circulating in a circuit. A solution with a closed window is described below.

In other embodiments, the cooling system can be configured to remove heat from the window 11.1 during treatment, so that said window is maintained at a low temperature and can cool through contact the epidermis, on which it is applied. For this purpose, the frame 11.2 in which the sapphire block 11.3 (or other suitable material forming the window 11.1) is inserted and retained can be made of a material having a high thermal conductivity and in heat exchange relationship with heat removing means. For example, the frame 11.2 can be equipped with ducts for the circulation of a cooling fluid. In other embodiments, the frame 11.2 can be in thermal contact with a heat exchanger, in which a cooling fluid circulates.

For example, the frame 11.2 can be made in one piece with, or welded to, a plate 18, in contact with a heat exchanger 21, in which a cooling liquid, such as water, circulates. Reference number 23 indicates a circuit for the circulation of the cooling liquid from a refrigeration device 25, which can be housed in the apparatus 3, to the handpiece 5 and vice versa. The cooling liquid that circulates in the circuit 23 extracts heat from the heat exchanger 21 and consequently, indirectly and through heat conduction, from the plate 18 and through this from the frame 11.2 in which the sapphire block 11.3 is mounted. The good thermal conductivity of the sapphire allows heat to be extracted by conduction from the surface of the epidermis E with which the sapphire block 11.3, defining the window 11.1 through which the laser beam F acting on the epidermis E passes, is in contact.

In other embodiments, not shown, cooling can take place through a thermoelectric device, for example through one or more Peltier cells, which can be arranged with the cold side in direct or indirect thermal contact with the frame 11.2 and with the hot side in heat exchange relationship with a heat removal system, for example with a heat exchanger 21, or with a ventilation system or the like.

Regardless of the cooling system, the epidermis can be cooled therewith before and preferably during treatment, in order to prevent overheating, a burning sensation felt by the patient and hence unwanted effects such as skin burns. Cooling at low temperatures with respect to the basal temperature, for example at temperatures of 2-15° C., also has an anesthetic effect, making the application of topical anesthetics to the epidermis unnecessary.

The window 11.1 can be positioned at a certain distance from the scanning system 17. The propagation space S of the laser beam from the scanning system 17 to the window 11.1 can be open. In this way, the operator can directly view the area of epidermis E treated through the window 11.1, so as to position the handpiece correctly and select the adequate scanning pattern. In some embodiments the laser beam propagation space S can be protected by a guard, preferably a movable guard 27. This guard can be provided with a movement of extension and retraction, schematically indicated with 127, for example controllable through a control button on the handle 13 of the handpiece. In other embodiments, the retraction movement can be controlled automatically, as a function of the conditions of emission or non-emission of the laser beam F by the laser source 7.

For example, it is possible to provide an opening and closing system of the movable guard 27 controlled by a button, in which opening is subordinate to a consent signal coming from the central control unit 9, so that opening is only allowed when no laser beam F is present between the scanning system 17 and the window 11.1. In other embodiments, the central control unit 9 can control automatic opening and closing of the movable guard 27 based on a safety criterion; the movable guard 27 remains closed during delivery of the laser beam F and is opened when the laser beam F is not emitted, so as to allow viewing of the area treated in safe conditions.

Through the movable guard 27 the operator can view the area of epidermis E treated in conditions of maximum safety, avoiding risks of burns to the hands or eyes, for example.

In some embodiments, the handpiece 5 can contain a video camera and/or a thermal camera, schematically indicated with 26, oriented so as to frame the area of epidermis being treated through the window 11.1. The image can be shown on a monitor, for example the monitor of the apparatus 3, or a separate monitor. The video camera 26 allows the operator to see the treatment field, suitably enlarged if necessary. Moreover, it allows the guard 27 of the path of the laser beam in the propagation space S to be kept closed. With this solution, it is possible to protect those present in the treatment room, namely the operator, the patient and any assistants, as well as the surrounding objects or furnishings, from the laser light. Advantageously, during the laser emission times, the video camera 26 can be protected from the light back-scattered by the applicator and/or by the skin, by means of a protective system synchronized with emission of the laser beam. In some embodiments, a mechanical shutter can be provided for this purpose. Preferably, in view of the brevity of the exposure time, an optical shutter or an electric control can be provided on the circuits of the optical sensor of the video camera, which contributes to forming the images. Using a thermal camera, it is also possible to monitor the temperature of the epidermis in real time.

The device 1 described above is used to perform a series of treatments, in partitular of cosmetic nature, such as treatments for reducing or eliminating wrinkles, non-ablative photo-rejuvenation treatment of the skin, treatments for removing benign pigmented marks (benign pigmented lesions) and treatments of atrophic scars, for example acne scars. More details on the mechanism of action will be provided below with reference to experimental results. In general, specifically referring to the treatment of skin wrinkles, the device is characterized by an interaction effect with the collagen fibers, due to the high affinity of the wavelength selected with these tissues, i.e., the high absorption coefficient of the collagen with respect to radiations with wavelengths in the selected range. The treatment of pigmented lesions draws advantage from the high affinity of the wavelength selected with melanin, i.e., the high absorption coefficient of melanin at these wavelengths.

Treatments performed with the device 1, using the emission spectrum defined above, are particularly effective due to the high affinity of the laser radiation used with melanin and with collagen fibers, i.e., the high absorption by the skin, by the melanin and by the collagen fibers, combined with a minimum affinity with the vascular component, i.e. a minimum absorption by the blood, contrary to what occurs with prior art devices, for example those operating in the near infrared range.

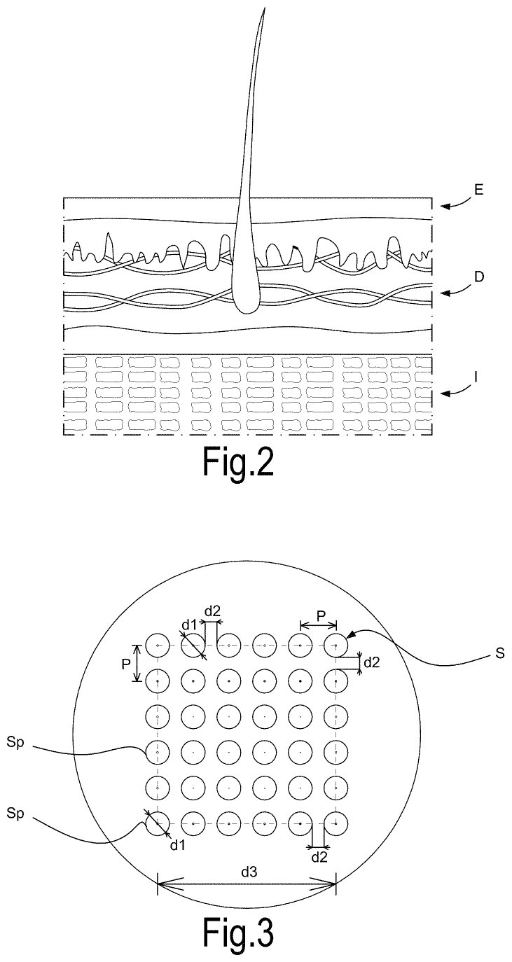

The advantages of the device disclosed herein, and of the methods implementable therewith, can be better understood with reference to the accompanying FIG. 2, which shows a cross-sectional view of a skin portion. As schematically indicated in FIG. 2, the skin consists of three layers: the epidermis E (surface skin layer), with the stratum corneum; the dermis D (intermediate skin layer), rich in small blood vessels and nerve endings; the hypodermis I (subcutaneous layer), with larger blood vessels.

The dermis D is rich in collagen fibers. External factors. such as solar radiation and aging cause a loss of the balance between collagen synthesis by the fibroblasts in the dermis and degradation thereof caused by enzymes of proteolytic action (matrix metalloproteinases). Enzymatic degradation of collagen causes a destruction of the protein chains that form collagen, resulting in the formation of shorter fragments of protein, without mechanical properties. With aging and, in some cases, due to factors such as exposure to ultraviolet radiation, an imbalance occurs between collagen production and the degradation of mature collagen. This imbalance eliminates the supporting function of collagen with respect to the extra-cellular matrix resulting in atrophy and loss of tone of the matrix.

Irradiation of the dermis by means of a laser beam in the red range, due to the selective affinity with collagen, stimulates the production of collagen without the occurrence of side effects typical of NIR lasers (the target of which is water) currently used for this type of treatments, effects that cause the formation of MEND (Microscopic Epidermal Necrotic Debris). Moreover, dermal-epidemial detachment, usually resulting from treatment with NIR laser for similar purposes, is also avoided. This results in a less invasive treatment and much faster recovery times compared to treatments with wavelengths in the infrared range. Moreover, there are also psychological advantages and advantages from a viewpoint of social relationships. In fact, treatment with laser in the red range does not cause visible alterations of the epidermis, with the exception of slight reddening that disappears rapidly (in a few tens of minutes) after treatment.

In order to optimize the treatment, the epidermis is preferably cooled with the cooling system to a temperature between about 2° C. and about 20° C., preferably between about 4° C. and about 15° C., typically about 5° C., for the purposes indicated above of reducing the burning sensation and possible local anesthetic effect. In the example illustrated, the cooling temperature is the temperature to which the material forming the window 11.3 is taken.

The power of the laser beam used can be between about 0.5 W and about 20 W, preferably between about 0.5 W and about 10 W.

The treatment can be of fractional type, i.e., performed by making the laser beam

F act on adjacent, but not overlapping, portions of epidermis. The treatment can be performed moving the laser beam F by means of the scanning system 17, so as to direct the laser beam F sequentially on portions of skin arranged according to a previously set pattern. FIG. 3 shows a treatment pattern according to areas arranged at nodes of an array with quadrangular mesh. Sp indicates the single spots, i.e. the single laser dots, acting on the surface of the epidermis E. The spots Sp can have a circular shape, with a diameter di that can be between about 0.1 mm and about 3 mm, preferably between about 0.5 mm and about 2 mm, more preferably between about 0.8 mm and about 1.2 mm, for example about 1 mm, or between about 0.6 mm and about 0.9 mm. In general, the spots have a diameter substantially larger than the diameter of prior art laser beams that use infrared radiations, with consequent benefits in terms of reducing the negative effects of the treatment.

The pitch P between the spot Sp can in general be selected so as to have a suitable distance between the spots Sp on the epidermis. With reference to FIG. 3, the distance between the spots is indicated with d2. This distance can, for example, be between 0 mm and about 4 mm, preferably between 0 and about 2 mm, more preferably between 0 and about 1.5 mm. Examples of applications of the treatment will be mentioned below, with indication of the distance d1. In some applications, the distance d2 can be zero and if necessary there can even be partial overlapping of successive spots Sp. In other applications, the spots are spaced from one another, so as to have a treatment of the epidermis of fractional type.

With a treatment of fractional type, it is possible to ensure that at the end of scanning of the area to be treated, the total surface of the epidermis that has been irradiated by the laser radiation is a fraction of the total of the surface of the area treated. Typically, it is possible, for example, to treat a quadrangular area, in which single spots or dots, on which the laser beam is directed over time by means of scanning of the laser beam, are identified. The percentage of irradiated surface (i.e., the sum of the area of the spots) with respect to the total of the area treated (in the case in hand, the area of the quadrangular surface) can, for example, be between about 2% and about 90%, preferably between about 5% and around 80%.

The laser emission can be continuous or pulsed. For example, in each position of the beam in the treatment pattern a single laser pulse or a plurality of laser pulses can be delivered.

The dwell time of the laser beam on each position of the pattern can be between about 0.01 seconds and about 2 seconds, preferably between about 0.01 seconds and about 1 second, even more preferably between 0.01 seconds and 0.5 seconds.

The dwell time can be divided into one or more application ranges, in the sense that in each point or spot one or more stacks, each having its own duration, can be emitted in sequence. The total dwell time on each treatment point is given by the sum of the durations of the single stacks. The dwell times indicated above can refer to the single stack. In other embodiments, the dwell times indicated can refer to the sum of the dwell times of each stack. As a function of the type of treatment and of other factors, for example the phototype (which can be relevant also for the definition of other emission parameters), the number of stacks can, for example, be between 1 and 10, preferably between 1 and 5.

Based on the power of the laser beam, on the total dwell time, or the number of stacks and the duration of each stack, and as a function of the size of the spot, it is possible to determine appropriate values of energy applied to each spot and of energy fluence per spot (energy per surface unit treated).

In some embodiments, the energy applied to each surface, i.e., spot or dot treated and for each single stack, can be between about 0.1 Joules and about 10 Joules, preferably between about 0.25 Joules and about 5 Joules.

As a function of the size of the area of each spot, in embodiments disclosed herein delivered fluence (i.e., energy per surface unit: J/cm²) ranges can be defined. In some embodiments, the fluence can be between about 51 J/cm² and about 2550 J/cm², preferably between about 100 J/cm² and about 1100 J/cm².

The parameters used can be selected not only as a function of the type of treatment to be performed, as will be clarified below with reference to some exemplary embodiment, but also based on the patient's phototype. The wavelength used and the range of power parameters, dwell times, number of stacks, energy and delivered fluence have proved particularly advantageous especially in the treatment of Asian skin phototypes.

The treated areas can have a total size d3×d3, for example, of less than 20×20 mm preferably less than 15×15mm. Alternatively, the areas treated can have an approximately circular shape, with a diameter, for example, equal to or less than around 20 mm, preferably equal to or less than 15 mm. Based on the size of the treated areas, on the size (for example the diameter) of the spots and on the distance between spots, the number of spots per treated area can be variable, for example, between about 30 and about 500, or between about 30 and about 70, or between about 120 and about 500.

In general, the greater the distance between single spots is, the smaller the percentage of surface impacted by the laser radiation with respect to the total treatment surface, i.e., the total surface on which the spots are distributed, will be. Therefore, the greater the distance is, the greater the fractional effect of the treatment will be. Typically, as indicated above, the percentage of surface irradiated by laser radiation with respect to the total can vary between around 2% and around 90%, preferably between around 5% and around 80%.

As will be clarified below with reference to examples of application, the aforesaid parameters can vary in narrower ranges defined by the type of skin imperfections to be treated.

The treatment can be suitably repeated for a number of sessions variable from 1 to 10, preferably from 1 to 5, more preferably from 2 to 4. The treatment sessions can be spaced by a recovery time between 2 weeks and two months, preferably between three weeks and six weeks, even more preferably around 1 month.

Experimental tests were conducted as set forth below for the treatment of various skin imperfections.

Treatment of Wrinkles:

In an embodiment the following parameters were used:
power of the laser beam 10 W
dwell time of the laser beam in each spot of the treatment pattern: from 200 to 250 ms
distance d2 between the treatment spots: from 1 to 1.5 mm
cooling temperature: 5° C.

Treatment of Benign Pigmented Lesions:

In an embodiment the following parameters were used:
power of the laser beam 10 W
dwell time of the laser beam in each spot of the treatment pattern: from 100 to 150 ms
distance d2 between the treatment spots: 0
cooling temperature: 5° C.

Fine Modification Treatment of the Texture of the Skin:

In an embodiment the following parameters were used:
power of the laser beam 10 W
dwell time of the laser beam in each spot of the treatment pattern: from 125 to 175 ms
distance d2 between the treatment spots: from 1 to 1.5 mm
cooling temperature: 5° C.

Treatment of Acne Scars:

In an embodiment the following parameters were used:
power of the laser beam 10 W
dwell time of the laser beam in each spot of the treatment pattern: from 300 to 400 ms
distance d2 between the treatment spots: from 1 to 1.5 mm
cooling temperature: 5° C.
   where cooling temperature refers to the temperature of the window of the handpiece in contact with the epidermis.

The data indicated above concern some values usable for some reference parameters. More in general, in embodiments parameters in the ranges of values indicated in the following table can be used for the specific applications mentioned:

| | wrinkles | | pigmented lesions | | texture | | acne scars | |
|---|---|---|---|---|---|---|---|---|
| | from: | to: | from: | to: | from: | to: | from: | to: |
| Power (W) | 5 | 10 | 5 | 10 | 5 | 10 | 5 | 10 |
| Dwell time (ms) | 125 | 150 | 75 | 150 | 100 | 100 | 125 | 200 |
| Stack | 1 | 2 | 1 | 1 | 1 | 2 | 1 | 2 |
| Energy per dot | 0.625 | 3 | 0.375 | 1.5 | 0.5 | 2 | 0.625 | 4 |
| (J) | | | | | | | | |
| dot size (mm) | 0.7 | 0.7 | 0.7 | 0.7 | 0.7 | 0.7 | 0.7 | 0.7 |
| dot area (cm²) | 0.003847 | 0.003847 | 0.003847 | 0.003847 | 0.003847 | 0.003847 | 0.003847 | 0.003847 |
| fluence per dot | 162.4854 | 779.9298 | 97.49123 | 389.9649 | 129.9883 | 519.9532 | 162.4854 | 1039.906 |
| (J/cm²) | | | | | | | | |
| area size (mm) | 15 | 15 | 15 | 15 | 15 | 15 | 15 | 15 |
| dot distance | 1.5 | 1.5 | 0 | 0.5 | 1.5 | 1.5 | 1.5 | 1.5 |
| (mm) | | | | | | | | |
| number of dots | 46.4876 | 46.4876 | 459.1837 | 156.25 | 46.4876 | 46.4876 | 46.4876 | 46.4876 |
| (N) | | | | | | | | |
| Cooling | 5 | 5 | 5 | 5 | 5 | 5 | 5 | 5 |
| temperature | | | | | | | | |
| (° C.) | | | | | | | | |
| Irradiated area | 0.079473 | 0.079473 | 0.785 | 0.267118 | 0.079473 | 0.079473 | 0.079473 | 0.079473 |
| (%) | | | | | | | | |

### EXPERIMENTAL DATA

In particular, a campaign of experimental tests was conducted for the treatment of wrinkles by means of laser with wavelengths of 675 nm of non-ablative fractional type. The following parameters were used for the tests:

power: 10 W
dwell time of the laser beam in each scanning position (spot): 400 ms
distance of the centers of the surfaces treated: 0.5 mm
energy delivered for each spot: 4 Joules
size of the diameter of the spot: 0.7 mm
fluence per spot: 1040 J/cm²
size of the area treated (square): 15×15 mm
distance between spots: 0.5 mm
number of spots per area treated: 156
percentage of the surface of epidermis irradiated: 27%
cooling: 5° C.

To evaluate the effects of the treatment, two biopsies were taken respectively before the treatment and 45 days after the treatment. The samples for histological analysis were immediately fixed in 10% neutral formalin, dehydrated in ethanol, cleared in HistoClear, and embedded in paraffin.

For evaluation by optical microscope, sections of 4-5 micrometers were stained with Van Gieson red and Picrosirius red for the collagen and with Weigert Van Gieson for the elastin. Further information on dermal collagen can be obtained with Picrosirius red, which not only stains the collagen fibers, but increases the birefringence of the collagen. Moreover, using circularly polarized light, the collagen fibers can have different colors when observed. Some fibers are red, others orange, yet others yellow or also green. With this staining method and using circularly polarized light it is also possible to distinguish between old and newly formed collagen. The former is red-orange, while the second is green-yellow.

Finally, the original images obtained by optical microscope were subjected to binary segmentation (using ImageJ, NIH).

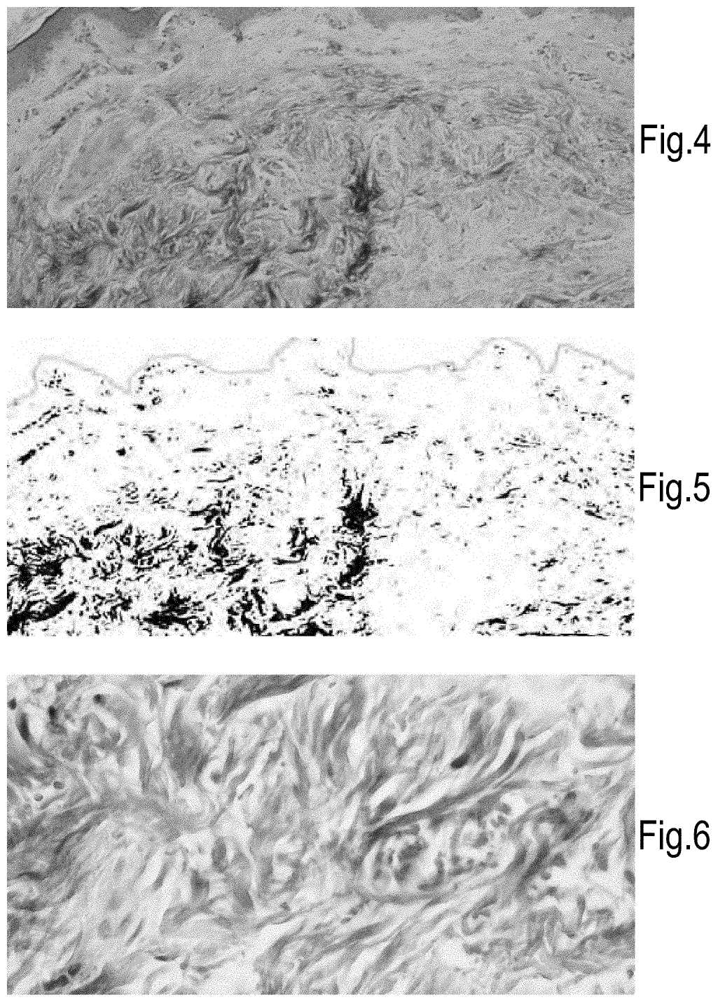

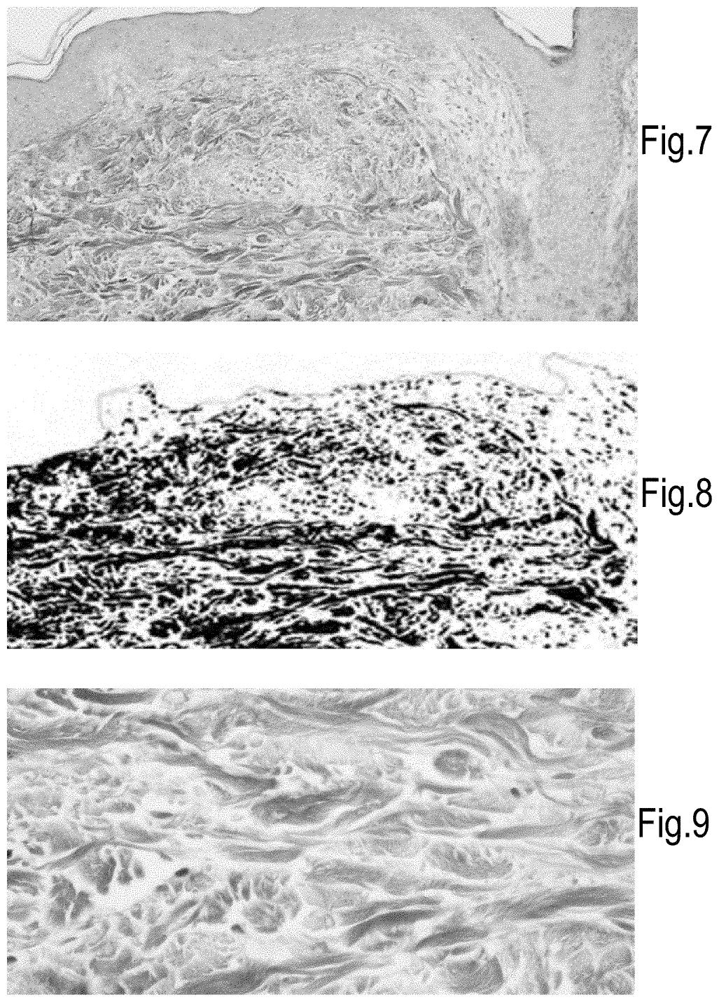

FIGS. 4 to 15 illustrate by way of example the efficacy of the treatment. More specifically, FIGS. 4, 5 and 6 show histological images obtained before treatment and FIGS. 7, 8 and 9 show similar histological images after treatment, of samples stained with Van Gieson stain (FIGS. 4, 6, 7 and 9) and subsequently obtained from binary segmentation

(FIGS. 5 and 8). More specifically, FIGS. 5 and 8 are obtained from binary segmentation of FIGS. 4 and 7. FIGS. 6 and 9 show the same samples as FIGS. 4 and 7 but twice enlarged.

By comparing FIGS. 4, 5, 6 and FIGS. 7, 8 and 9 it can be seen that after treatment the reticular layer (deep dennis) has a decreased compactness of the collagen fibers, no bands of collagen fibers and the presence of finer, more parallel and more linear fibers, compared to the images before treatment.

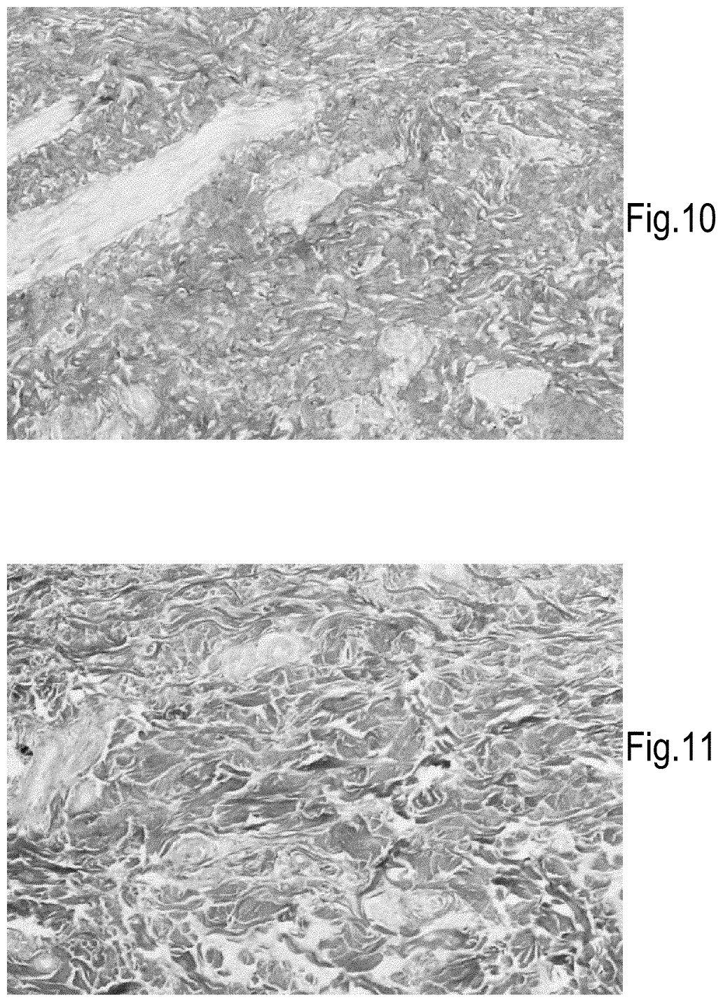

FIGS. 10 and 11 show histological images obtained on samples before (FIG. 10) and after (FIG. 11) treatment. The images were obtained by staining with Picrosirius red. Before treatment, the connective tissue of the reticular dermis is deeply stained (fibers in bands). After treatment, the connective tissue is mainly formed of thin fibrils.

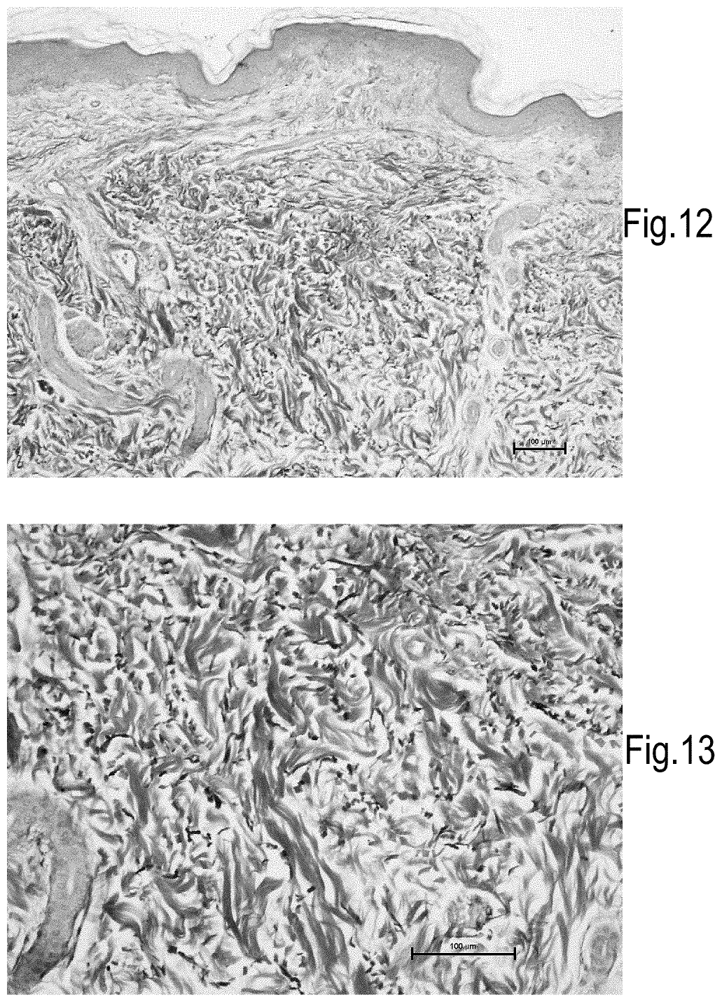

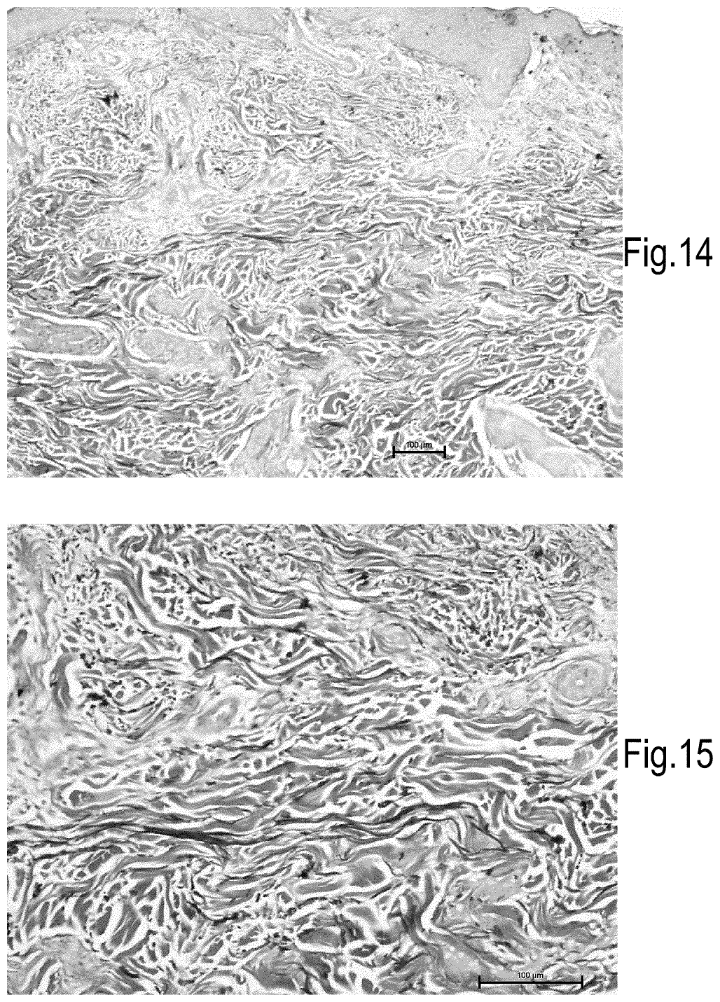

FIGS. 12 and 13 show histological images before treatment and FIGS. 14 and 15 show histological images after treatment obtained from samples stained with Weigert Van Gieson stain. The treatment caused the elastic fibers (dark fibers) to be more parallel and straighter in the dermis after treatment.

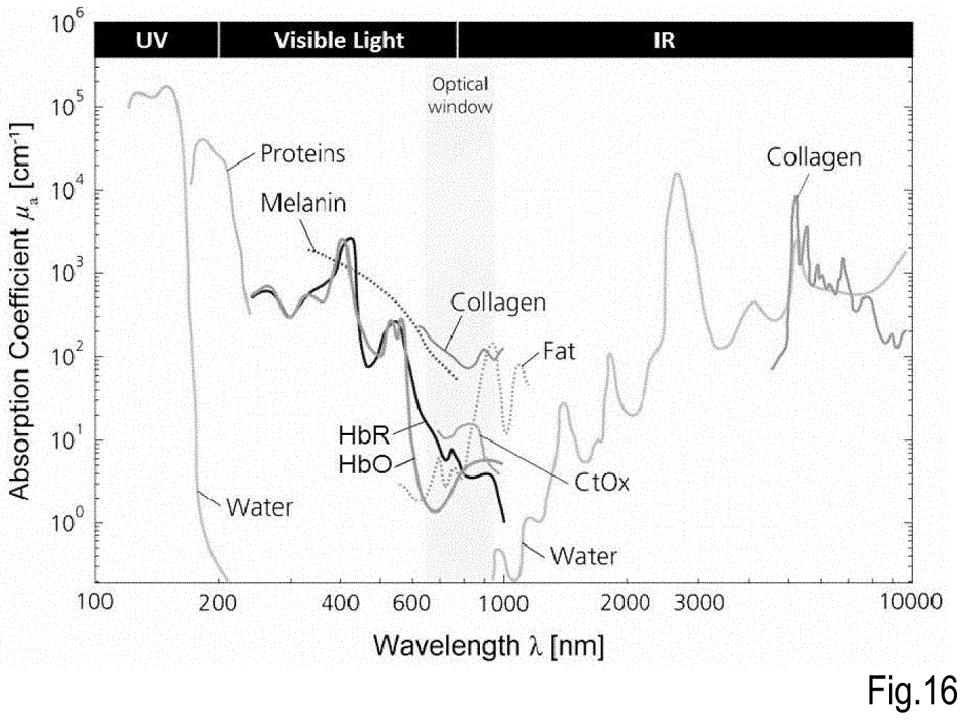

The results set forth above were obtained with a source at 675 nm, the radiation of which is absorbed selectively by the collagen while it has poor absorption by other components present in the tissues treated, which allows the energy to be conveyed in a precise and targeted manner toward the collagen. This effect can be obtained in a range of wavelengths in the red spectrum. FIG. 16 illustrates the absorption diagram of the various components as a function of wavelength. More specifically, the various curves show the trend of the absorption coefficient (in cm⁻¹) as a function of wavelength (in nanometers, nm) for the following chromophores: water, proteins, melanin, HbR (deoxyhemoglobin), HbO (oxyhemoglobin), collagen, CtOx (cytochrome oxidase), adipose cells.

The range of wavelengths selected allows maximization of absorption by the collagen fibers, minimizing absorption by other chromophores present. Specifically, although collagen has absorption peaks also at wavelengths around 6000 nm, this wavelength was discarded as coincident with an absorption peak of water. Wavelengths close to 600 nm and close to 1000 nm are somewhat ineffective as they cause marked absorption by the melanin and by the adipose cells, respectively.

The device and the method described allow the implementation of a non-invasive procedure that stimulates the production of dermal collagen and strengthens the elastin fibers. This gives rise to a significant increase in new thin collagen fibers, resulting in an increase in dermal thickness. The wavelength, power and density used, also due to the simultaneous cooling of the surface of the skin, prevent epidermal alterations and inflamoratory reactions from occurring.

1. A device for skin treatment, the device comprising:
a laser source adapted to emit a laser radiation at a wavelength between about 620 nm and about 750 nm;
a handpiece, comprising: an applicator with an epidermis contacting surface defining a window for the passage of a laser beam toward the epidermis;
a waveguide adapted to convey laser radiation from the laser source to the handpiece.

2. The device of claim 1, comprising a cooling system, preferably in heat exchange relationship with the epidermis contacting surface.

3. The device of claim 1, further comprising a scanning system.

4. The device of claim 1, wherein the laser source is adapted to emit a wavelength between around 635 nm and around 715 nm.

5. The device of claim 1, wherein the epidermis contacting surface comprises a window made of sapphire.

6. The device of claim 1, wherein the handpiece comprises: a handle, in which the scanning system is housed; a spacer interposed between the scanning system and the epidermis contacting surface; a heat transfer device from the epidermis contacting surface to the handpiece; an optical path from the scanning system to the epidermis contacting surface.

7. The device of claim 6, wherein the handpiece comprises a removable protection device associated with the optical path.

8. The device of claim 6, wherein the handpiece comprises a path for a cooling fluid in heat exchange relationship with a cooling plate, adapted to remove heat from the epidermis contacting surface.

9. The device of claim 1, comprising a video camera, preferably integrated in the handpiece, adapted to frame a treatment area, preferably through the window for the passage of the laser beam toward the epidermis.

10. The device of claim 9, wherein the video camera is associated with a protection system synchronized with the laser emission.

11. The device of claim 1, comprising a laser radiation collimator configured to generate a laser spot with a size between about 0.1 and about 3 mm.

12. The device of claim 1, wherein the laser source emits at a power between about 0.5 W and about 20 W.

13. The device of claim 1, wherein the scanning system is configured to selectively and sequentially irradiate portions of epidermis according to a given pattern, and wherein the device is controlled so as to have a dwell time of the laser beam for each portion of epidermis between about 0.01 seconds and about 1 second.

14. The device of claim 1, wherein the scanning system is configured to selectively and sequentially irradiate portions of epidermis according to a given pattern, such that the laser spots on the surface of the epidermis are spaced with respect to one another by from 0 mm to about 4 mm.

15. The device of claim 1, wherein the scanning system and the laser source are controlled so as to perform a fractional treatment, wherein the percentage of surface of epidermis irradiated is between about 2% and about 90% of the total treatment surface.

16. The device of claim 15, wherein the treatment surface has a maximum size between about 10 mm and about 25 mm.

17. The device of claim 16, wherein the scanning system is controlled to irradiate a number of spots between 30 and in the treatment surface.

18. The device of claim 1, controlled to emit, in each position of a scanning pattern, a number of stacks from 1 to 10.

19. The device of claim 1, wherein the laser source is controlled to irradiate a dose of energy between about 0.1 Joules and about 10 Joules, with a dwell time between about 0.01 seconds and about 2 seconds for each spot.

20. The device of claim 19, wherein the size of the laser beam spot is such as to obtain a fluence between about 51 Joules/cm² and about 2550.

21. A method for non-invasive and non-ablative cosmetic treatment for removing or reducing skin imperfections, the method comprising the step of irradiating a portion of epidermis of the subject being treated with a laser beam at a wavelength between about 620 nm and about 750 nm.

22. The method of claim 21, comprising the step of cooling the portion of epidermis before and/or during irradiation by means of the laser beam.

23. The method of claim 21, wherein the laser beam has a power between about 0.5 W and about 20 W.

24. The method of claim 21, comprising the step of sequentially irradiating with the laser beam portions of epidermis according to a set pattern.

25. The method of claim 21, comprising the following steps:
applying a handpiece to the epidermis of the patient;
conveying the laser beam toward the handpiece;
delivering the laser beam to the epidermis by means of the handpiece;
cooling the epidermis in the area of application of the handpiece before and/or during delivery of the laser beam.

26. The method of claim 21, wherein the step of delivering the laser beam comprises the step of scanning the laser beam by means of a scanning system and directing the laser beam sequentially in spots of the epidermis according to a set pattern.

27. The method of claim 26, wherein the spot of the epidermis irradiated sequentially are spaced with respect to one another at a distance between 0 mm and about 4 mm.

28. The method of claim 21, wherein the treatment surface has a maximum size between about 10 mm and about 25 mm.

29. The method of claim 21, comprising the step of irradiating a number of spots between 30 and 500.

30. The method of claim 21,
wherein in each of a plurality of scanning positions of the laser beam the laser beam is applied in sequence from 1 to 10 times in time sequence.

31. The method of claim 21, wherein in each scanning position at least a dose of energy between about 0.1 Joules and about 10 Joules.

32. The method of claim 31, wherein the size of the laser beam spot is such as to obtain a fluence between about 51 Joules/cm2 and about 2550 Joules/cm².

33. The method of claim 21, wherein the skin imperfection comprises one or more of the following: wrinkles, benign pigmented lesions (pigmented marks), atrophic scars.
